(19) United States
(12) Patent Application Publication
(10) Pub. No.: US 2026/0183366 A1
(43) Pub. Date: Jul. 2, 2026

(54) LIQUID DALBAVANCIN COMPOSITION

(71) Applicant: Hikma Pharmaceuticals USA Inc., Berkeley Heights, NJ (US)

(72) Inventors: Martina Manenica, Zagreb (HR); Ema Kovacevic, Zagreb (HR); Ernest Mestrovic, Zagreb (HR)

(73) Assignee: Hikma Pharmaceuticals USA Inc., Berkeley Heights, NJ (US)

(21) Appl. No.: 19/116,550

(22) PCT Filed: Oct. 11, 2023
(86) PCT No.: PCT/EP2023/078261

§ 371 Date: Mar. 28, 2025

Related U.S. Application Data
(60) Provisional application No. 63/518,022, filed on Aug. 7, 2023.

(30) Foreign Application Priority Data
Oct. 12, 2022 (DK) PA202200932
Feb. 17, 2023 (EP) 23157325.4

(51) Int. Cl. A61K 38/16; A61K 47/12; A61K 47/18; A61K 47/22; A61K 47/26

(52) U.S. Cl. CPC A61K 38/164; A61K 47/12; A61K 47/183; A61K 47/22; A61K 47/26

(57) ABSTRACT

The present disclosure relates to a stable aqueous composition of dalbavancin, the process for making such compositions and use of such compositions for treatment of a patient in need thereof.

### CROSS-REFERENCE TO RELATED APPLICATIONS

This application is a bypass continuation of PCT/EP2023/078261 filed on Oct. 11, 2023, which claims priority to Denmark Application No. PA202200932 filed Oct. 12, 2022, EP Application Serial No. 23157325.4 filed Feb. 17, 2023, and U.S. Provisional Application No. 63/518,022 filed on Aug. 7, 2023, which are incorporated herein by reference in their entirety.

### FIELD OF THE INVENTION

The present disclosure relates to stable aqueous composition of dalbavancin, the process for making such compositions and use of such compositions for treatment of a patient in need thereof. Such compositions provide good stability.

### BACKGROUND

Dalbavancin is a semisynthetic lipoglycopeptide and exerts its bactericidal effect by disrupting cell wall biosynthesis. It binds to the D-alanyl-D-alanyl residue on growing peptidoglycan chains and prevents transpeptidation from occurring, preventing peptidoglycan elongation and cell wall formation.

Dalbavancin is manufactured by fermentation of a selected Nonomuraea strain to generate the natural glycopeptide complex A-40926. This precursor is then selectively esterified at the carboxyl group of its sugar moiety, its peptidyl carboxyl group is amidated and the ester of the N-acylaminoglucuronic acid carboxyl group is saponified. The outcome is a compound mixture of two closely related structural families—A and B—that can be further subdivided into a total of five subtypes (Table 1).

The structure of dalbavancin is shown below:

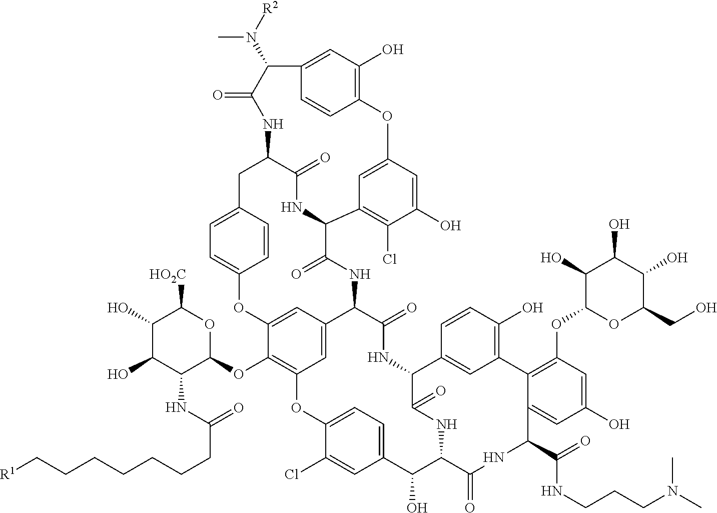

TABLE 1
Dalbavancin subtypes

| | Alkyl sidechain of N- | Amino-terminal |
|---|---|---|
| Homolog | acylaminoglucuronic acid (R¹) | substituent (R²) |
| A₀ | CH(CH₃)₂ | H |
| A₁ | CH₂CH₂CH₃ | H |
| B₀ | CH₂CH(CH₃)₂ | H |
| B₁ | CH₂CH₂CH₂CH₃ | H |
| B₂ | CH₂CH(CH₃)₂ | CH₃ |

Dalbavancin is marketed under the tradename DALVANCE® in US and XYDALBA® in Europe. The marketed product is a lyophilized powder containing dalbavancin hydrochloride, lactose monohydrate and mannitol. It may also contain sodium hydroxide and/or hydrochloric acid. The lyophilized powder needs to be reconstituted and diluted prior to administration to a patient. The package insert for DALVANCE® (dalbavancin) for injection instructs the user to use either Sterile Water for Injection, USP, or 5% Dextrose Injection, USP, for reconstitution of the lyophilized product and subsequently to dilute only with 5% Dextrose Injection, USP, to a final concentration of 1 mg/mL to 5 mg/ml.

The total time from reconstitution to dilution to administration should not exceed 48 hours.

Dalbavancin is marketed for treatment of adult and pediatric patients with acute bacterial skin and skin structure infections (ABSSSI) caused by designated susceptible strains of Gram-positive microorganisms.

What is needed are stable liquid compositions of dalbavancin that do not need reconstitution and/or dilution prior to administration and are stable over a long period of time.

### SUMMARY

It has been found that the liquid compositions of dalbavancin described herein possess surprisingly improved stability. In particular, it has been found certain liquid compositions that are stable for a certain period of time at room temperature.

It has been found that an aqueous formulation of dalbavancin and optionally an osmolality adjusting agent, in water with a pH in the range of 4.8 to 5.9 is stable for a certain period of time at room temperature.

It has also been found that these aqueous formulations of dalbavancin will have low levels of mannosyl aglycon impurity.

It has been found that these aqueous formulations of dalbavancin have good chemical and physical stability for a certain period of time at room temperature.

### DETAILED DESCRIPTION (INCLUDING DEFINITIONS)

As used herein, the terms “pharmaceutical composition”, “pharmaceutical formulation”, “composition” and “formulation” are used interchangeably.

By the term “aqueous solution” is understood any solution in which water is present at or above 50% v/v, such as, e.g. a solution comprising from about 50% v/v to about 100% v/v water. Accordingly, aqueous solutions include solutions comprising about 50% v/v or more, about 60% v/v or more, about 70% v/v or more, about 75% v/v or more, about 80% v/v or more, about 85% v/v or more, about 90% v/v or more, about 95% v/v or more or about 100% v/v water.

“Parenteral administration” includes, for example, subcutaneous injections, intravenous injections, intraperitoneal injections, intramuscular injections, intrasternal injections, and infusion. Injectable preparations (i.e., sterile injectable aqueous or oleaginous suspensions) may be formulated according to the known art using suitable dispersing, wetting, suspending agents and/or solubilizing agents.

The term “ready-to-administer” is synonymous with “ready-to-infuse” or “ready-to-inject” and is not to be read as the term “ready-to-use” aqueous solution.

The term “ready-to-use” includes aqueous preconcentrates which require a single step of dilution with an aqueous diluent fluid such as water for injection or saline before administration. The term “ready-to-administer” is also distinguished from lyophilized products that require two steps, a first step of reconstitution to form a preconcentrate and then a second step where the preconcentrate is subjected to dilution with an aqueous infusion fluid. The “ready-to-administer” parenteral dosage form according to the present disclosure avoids the inconvenience of reconstituting or diluting a concentrated parenteral formulation into infusion diluents prior to infusion, as well as eliminates the risk of any potential calculation or dilution error as well as risks of microbiological contamination during handling.

The aqueous dalbavancin formulations described herein may be a ready-to-use or a ready-to-administer solution that may be packed in a flexible plastic container or it may be packed in a vial or a bottle.

As used herein, the term “flexible plastic container” means flexible polymeric infusion bags or other polymeric containers. Exemplary flexible plastic containers are made of polyolefins, such as polyethylene, polypropylene, copolymers and derivatives thereof, with or without other additives.

Typically, the compounds of the present disclosure are administered in an amount effective to treat a condition as described herein. The compounds of the present disclosure are administered by any suitable route in the form of a pharmaceutical composition adapted to such a route, and in a dose effective for the treatment intended. Therapeutically effective doses of the compounds required to treat the progress of the medical condition are readily ascertained by one of ordinary skill in the art using preclinical and clinical approaches familiar to the medicinal arts.

The term “therapeutically effective amount” as used herein refers to that quantity of a compound or pharmaceutical composition that is sufficient to result in a desired activity upon administration to a subject in need thereof. An “effective amount” means the amount of a compound or pharmaceutical composition according to the present disclosure that, when administered to a patient for treating an infection or disease is sufficient to effect such treatment. The “effective amount” will vary depending on the active ingredient, the state of infection, disease or condition to be treated and its severity, and the age, weight, physical condition and responsiveness of the mammal to be treated.

Dalbavancin is prone to degradation and the most important impurity is the mannosyl aglycon (MAG) impurity shown below:

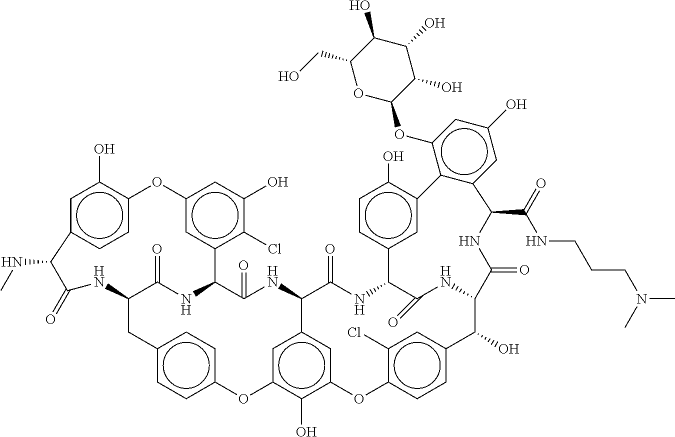

The term “stability”, “chemical stability” or “stable” means that the product, composition or formulation exhibits an acceptable amount of dalbavancin being present, or not more than a certain amount of dalbavancin has degraded after a certain period of time. Accordingly, in a stable product, solution or formulation, unacceptable degradation of the active agent is avoided.

Stability can be presented as the purity or assay of dalbavancin in a composition according to the disclosure. If the composition initially contains dalbavancin of a certain purity or assay, the stability of the composition will be reflected by a decrease in the same in the product, formulation or composition over time, where a stable composition would contain the dalbavancin of a specified chromatographic purity or assay after a predetermined time period. For example, the formation of MAG is reduced in a stable product.

For example, a stable composition can be one which has not more than a 1%, 2%, 3%, 4%, 5%, 6%, 7%, 8%, 9%, 10%, assay degrease/drop of dalbavancin after a predetermined time period analyzed by liquid chromatography, e.g., HPLC, UHPLC, or LC/MS.

Accordingly, “stability” may also be defined by the amount of total or individual impurities generated after a certain period of time. The amount of impurities being present may be expressed as a percentage, for example as a peak-area percentage of a HPLC chromatogram or calculated according to standard solution.

The degradation of dalbavancin to produce mannosyl aglycon impurity may be identified based on the relative retention time (RRT) of dalbavancin and mannosyl aglycon impurity in an HPLC chromatogram.

As used herein, the increase (delta) in mannosyl aglycon impurity is measured from the time of preparation of the formulation and storage through the specified time, e.g., 3 months and 6 months.

For example, a stable composition can be one which has not more than a 0.3%, 0.4%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, or a 2.0% increase in the amount of mannosyl aglycon impurity after storage at 25° C. for 3 months.

In addition to chemical stability, the physical stability of the composition may be monitored. Physical stability is defined as the appearance of the formulation and includes visual inspection of precipitation, clarity, and color of the solution.

Color can be determined spectrophotometrically by using the L*a*b* color space method and calculating the ΔE in accordance with USP <1061>.

In liquid ready-to-administer pharmaceutical products it is important to have formulations without any visible particles or precipitation.

In an aspect, the aqueous dalbavancin formulations according to the present disclosure are stable at a temperature of from 2° C. to 8° C. for a certain period of time. In an aspect, the aqueous dalbavancin formulations according to the present disclosure are stable under room temperature conditions for a certain period of time. By the term “room temperature” used herein, is meant from 20° C. to 27° C. In an aspect, the aqueous dalbavancin formulations according to the present disclosure are stable at 40° C. for a certain period of time. In an aspect, the aqueous dalbavancin formulations described herein are stable over time periods of 7 days (1 week), 14 days (2 weeks), 30 days (1 month), 60 days (2 months), 3 months, 4 months, 180 days (6 months), 9 months, 12 months (1 year), 14 months, 16 months, 18 months, 20 months, 24 months or more at certain specified temperature conditions.

The formulations disclosed herein may be sterilized by known means. Such known means in the art comprise, for example, sterile filtration.

The term “pharmaceutically acceptable salt” refers to a salt prepared by combining a compound of the present disclosure with an acid whose anion, or a base whose cation, is generally considered suitable for use in humans.

The term “dalbavancin” as used herein means dalbavancin or a pharmaceutically acceptable salt of dalbavancin. Dalbavancin is a mixture of two closely related structural compound families—A and B—that can be further subdivided into a total of five subtypes, as shown in the table above. B₀ is the main component of the mixture and the components A₀, A₁, B₁ and B₂ are present in lower amounts. Pharmaceutically acceptable salts of dalbavancin may be salts derived from inorganic or organic acids.

In one aspect, the pH of the liquid dalbavancin formulations is in the range from 4.8 to 5.9. In an aspect, the pH is in the range of 5.0 to 5.8. In another aspect the pH is in the range of 5.2 to 5.7 or in the range of 5.0 to 5.5. In another aspect the pH is 4.8, 4.9, 5.0, 5.1, 5.2, 5.3, 5.4, 5.5, 5.6, 5.7, 5.8 or 5.9.

“pH” is the conventional measurement unit of hydrogen ion activity in aqueous or other liquid solutions at room temperature, unless another temperature is specified.

In an aspect, pH values are given for the formulations just after preparation, which means at the start of the stability testing.

The pH of the formulation may be adjusted in any suitable manner. The pH may be adjusted with one or more pH adjusting agents, which may be selected from acids or bases. Examples of pH-adjusting agents include hydrochloric acid and sodium hydroxide and combinations thereof.

For the aqueous dalbavancin formulations it is important to keep the pH stable over time, e.g., to minimize the shift in pH over time. As used herein, the term “shift in pH” means the change of the pH in the formulation from immediately after the formulation has been made and the measured pH after a certain time as for example 3 months, 6 months, 9 months, 12 months, 16 months, 18 months, 24 months or longer.

Formulations with no or little shift in pH over time has improved chemical and physical stability.

In an aspect, the pH in the formulation does not shift more than 1.0 pH unit after storage at room temperature for at least 6 months.

In an aspect, the pH in the formulation does not shift more than 0.8 pH units after storage at room temperature for at least 6 months.

In an aspect, the pH in the formulation does not shift more than 0.6 pH units after storage at room temperature for at least 6 months.

For intravenous products to be acceptable for administration to humans, such products must have adequate osmolality.

The aqueous dalbavancin formulations described herein may optionally comprise an osmolality adjusting agent. The osmolality adjusting agent may be dextrose.

In an aspect, the osmolality adjusting agent is dextrose.

In an aspect, the concentration of an osmolality adjusting agent in the product is in the amount to provide an iso-osmotic ready-to-administer or ready-to-use product.

In an aspect, the aqueous dalbavancin formulations have an osmolality within the physiological osmolality of blood. According to the literature, and as used herein, the physiological osmolality of blood is in the range of 270 to 340 mOsmol/kg.

In an aspect, the concentration of an osmolality adjusting agent in the aqueous dalbavancin formulations should be in the amount to achieve an osmolality of the product within the targeted range of 270 to 340 mOsmol/kg.

In an aspect, the aqueous dalbavancin formulations are both isotonic and have an osmolality within the physiological osmolality of blood as described above.

As mentioned above it is important to keep the pH stable in the formulations over time. To decrease the shift in pH in the formulation an agent that stabilizes pH can be included in the formulation. It is also known that it is important to have an aqueous dalbavancin formulation without any visible particles.

By including at least one amino acid in the aqueous dalbavancin formulation we have seen that the shift in pH can be stabilized and/or help with the physical stability of the solution.

Aqueous dalbavancin formulations including at least one amino acid will have less shift in pH and thus have less degradation of dalbavancin and the formulation remains clear.

The term “amino acid” means any amino acid, including, but not limited to the 20 amino acids naturally occurring in peptides in both D and L-form and is also meant to cover any salt thereof, especially pharmaceutically acceptable salts. For example, the term “amino acid” includes Alanine, Arginine, Asparagine, Aspartic acid, Cysteine, Glutamic acid, Glutamine, Glycine, Histidine, Isoleucine, Leucine, Lysine, Methionine, Phenylalanine, Proline, Serine, Threonine, Tryptophan, Tyrosine, Valine and Ornithine, and any configurations thereof.

Thus, the term “amino acid” includes L-Alanine, L-Arginine, L-Asparagine, L-Aspartic acid, L-Cysteine, L-Glutamic acid, L-Glutamine, L-Histidine, L-Isoleucine, L-Leucine, L-Lysine, L-Methionine, L-Phenylalanine, L-Proline, L-Serine, L-Threonine, L-Tryptophan, L-Tyrosine, L-Valine and L-Ornithine.

Thus, included is D-Alanine, D-Arginine, D-Asparagine, D-Aspartic acid, D-Cysteine, D-Glutamic acid, D-Glutamine, D-Histidine, D-Isoleucine, D-Leucine, D-Lysine, D-Methionine, D-Phenylalanine, D-Proline, D-Serine, D-Threonine, D-Tryptophan, D-Tyrosine, D-Valine and D-Ornithine.

In an aspect, the amino acid is in L-form.

In an aspect, the formulation comprises one or more amino acids.

In an aspect, the formulation comprises one or more L-amino acids.

In an aspect, the aqueous dalbavancin formulations do not comprise a buffer.

In an aspect, the aqueous dalbavancin formulations do not comprise phosphate buffer.

In an aspect, the aqueous dalbavancin formulations do not comprise acetate buffer.

In an aspect, the aqueous dalbavancin formulations do not comprise citrate buffer.

The term “solubilizing agent” is an agent included in the formulation and that helps dalbavancin formulations to remain clear and no precipitate is seen in the formulations. A solubilizing agent can be organic molecules, for example the solubilizing agent may be polyethylene glycol (PEG) such as PEG 400.

In an aspect, the composition comprises dalbavancin hydrochloride.

In an aspect, the composition comprises dalbavancin hydrochloride and water.

The concentration of dalbavancin in the composition may be in the range of 1 mg/ml to 25 mg/ml. In an aspect, the concentration of dalbavancin is in the range of 1 mg/ml to 10 mg/l, 1 mg/ml to 7 mg/ml or 3 mg/ml to 6 mg/ml. In an aspect, the concentration of dalbavancin is in the range of 4 mg/ml to 6 mg/ml. In another aspect the concentration of dalbavancin is 4.0 mg/ml, 4.1 mg/ml, 4.2 mg/ml, 4.3 mg/ml, 4.4 mg/ml, 4.5 mg/ml, 4.6 mg/ml, 4.7 mg/ml, 4.8 mg/ml, 4.9 mg/ml, 5.0 mg/ml, 5.1 mg/ml, 5.2 mg/ml, 5.3 mg/ml, 5.4 mg/ml, 5.5 mg/ml, 5.6 mg/ml, 5.7 mg/ml, 5.8 mg/ml, 5.9 mg/ml or 6.0 mg/ml. In yet another aspect, the concentration of dalbavancin is 5 mg/ml. In an aspect, the concentration of dalbavancin is in the range of 10 mg/ml to 25 mg/ml. In an aspect the concentration of dalbavancin is in the range of 15 mg/ml to 22 mg/ml. In another aspect, the concentration of dalbavancin is in the range of 18 mg/ml to 22 mg/ml. In another aspect, the concentration of dalbavancin is 18 mg/ml, 19 mg/ml, 20 mg/ml, 21 mg/ml or 22 mg/ml.

When specific amounts or ranges of amounts of dalbavancin are given in this application, all values are calculated based on dalbavancin base.

In an aspect, the aqueous composition of dalbavancin comprising dalbavancin or a pharmaceutically acceptable salt of dalbavancin and water, wherein the pH is from 4.8 to 5.9.

In an aspect, the aqueous composition of dalbavancin comprising dalbavancin or a pharmaceutically acceptable salt of dalbavancin, an osmolality adjusting agent and water, wherein the pH is from 4.8 to 5.9.

In an aspect, the aqueous composition of dalbavancin comprising dalbavancin or a pharmaceutically acceptable salt of dalbavancin, an osmolality adjusting agent, one or more amino acid and water, wherein the pH is from 4.8 to 5.9.

In an aspect, the aqueous composition of dalbavancin comprising dalbavancin or a pharmaceutically acceptable salt of dalbavancin and water, wherein the pH is from 4.8 to 5.9 and wherein the concentration of dalbavancin is in the range of 1 mg/ml to 25 mg/ml.

In an aspect, the aqueous composition of dalbavancin comprising dalbavancin or a pharmaceutically acceptable salt of dalbavancin, an osmolality adjusting agent and water, wherein the pH is from 4.8 to 5.9 and wherein the concentration of dalbavancin is in the range of 1 mg/ml to 25 mg/ml.

In an aspect, the aqueous composition of dalbavancin comprising dalbavancin or a pharmaceutically acceptable salt of dalbavancin, an osmolality adjusting agent, one or more amino acid and water, wherein the pH is from 4.8 to 5.9 and wherein the concentration of dalbavancin is in the range of 1 mg/ml to 25 mg/ml.

In an aspect, the aqueous composition of dalbavancin comprising dalbavancin or a pharmaceutically acceptable salt of dalbavancin and water, wherein the pH is from 4.8 to 5.9 and wherein the concentration of dalbavancin is in the range of 15 mg/ml to 22 mg/ml.

In an aspect, the aqueous composition of dalbavancin comprising dalbavancin or a pharmaceutically acceptable salt of dalbavancin, an osmolality adjusting agent and water, wherein the pH is from 4.8 to 5.9 and wherein the concentration of dalbavancin is in the range of 15 mg/ml to 22 mg/ml.

In an aspect, the aqueous composition of dalbavancin comprising dalbavancin or a pharmaceutically acceptable salt of dalbavancin, an osmolality adjusting agent, one or more amino acid and water, wherein the pH is from 4.8 to 5.9 and wherein the concentration of dalbavancin is in the range of 15 mg/ml to 22 mg/ml.

In an aspect, the aqueous composition of dalbavancin comprising dalbavancin or a pharmaceutically acceptable salt of dalbavancin and water, wherein the pH is from 4.8 to 5.9 and wherein the concentration of dalbavancin is in the range of 1 mg/ml to 7 mg/ml.

In an aspect, the aqueous composition of dalbavancin comprising dalbavancin or a pharmaceutically acceptable salt of dalbavancin, an osmolality adjusting agent and water, wherein the pH is from 4.8 to 5.9 and wherein the concentration of dalbavancin is in the range of 1 mg/ml to 7 mg/ml.

In an aspect, the aqueous composition of dalbavancin comprising dalbavancin or a pharmaceutically acceptable salt of dalbavancin, an osmolality adjusting agent, one or more amino acid and water, wherein the pH is from 4.8 to 5.9 and wherein the concentration of dalbavancin is in the range of 1 mg/ml to 7 mg/ml.

In an aspect, the aqueous composition of dalbavancin consists essentially of dalbavancin or a pharmaceutically acceptable salt of dalbavancin and water, wherein the pH is from 4.8 to 5.9.

In an aspect, the aqueous composition of dalbavancin consists of dalbavancin or a pharmaceutically acceptable salt of dalbavancin and water, wherein the pH is from 4.8 to 5.9.

In an aspect, the aqueous composition of dalbavancin consists essentially of dalbavancin or a pharmaceutically acceptable salt of dalbavancin, an osmolality adjusting agent and water, wherein the pH is from 4.8 to 5.9.

In an aspect, the aqueous composition of dalbavancin consists essentially of dalbavancin or a pharmaceutically acceptable salt of dalbavancin, an osmolality adjusting agent, one or more amino acid and water, wherein the pH is from 4.8 to 5.9.

In an aspect, the aqueous composition of dalbavancin consists of dalbavancin or a pharmaceutically acceptable salt of dalbavancin, an osmolality adjusting agent and water, wherein the pH is from 4.8 to 5.9.

In an aspect, the aqueous composition of dalbavancin consists of dalbavancin or a pharmaceutically acceptable salt of dalbavancin, an osmolality adjusting agent, one or more amino acid and water, wherein the pH is from 4.8 to 5.9.

In an aspect, the aqueous composition of dalbavancin consists essentially of dalbavancin or a pharmaceutically acceptable salt of dalbavancin and water, wherein the pH is from 4.8 to 5.9 and wherein the concentration of dalbavancin is in the range of 1 mg/ml to 25 mg/ml.

In an aspect, the aqueous composition of dalbavancin consists of dalbavancin or a pharmaceutically acceptable salt of dalbavancin and water, wherein the pH is from 4.8 to 5.9 and wherein the concentration of dalbavancin is in the range of 1 mg/ml to 25 mg/ml.

In an aspect, the aqueous composition of dalbavancin consists essentially of dalbavancin or a pharmaceutically acceptable salt of dalbavancin and water, wherein the pH is from 4.8 to 5.9 and wherein the concentration of dalbavancin is in the range of 1 mg/ml to 7 mg/ml.

In an aspect, the aqueous composition of dalbavancin consists of dalbavancin or a pharmaceutically acceptable salt of dalbavancin and water, wherein the pH is from 4.8 to 5.9 and wherein the concentration of dalbavancin is in the range of 1 mg/ml to 7 mg/ml.

In an aspect, the aqueous composition of dalbavancin consists essentially of dalbavancin or a pharmaceutically acceptable salt of dalbavancin and water, wherein the pH is from 4.8 to 5.9 and wherein the concentration of dalbavancin is in the range of 15 mg/ml to 22 mg/ml.

In an aspect, the aqueous composition of dalbavancin consists of dalbavancin or a pharmaceutically acceptable salt of dalbavancin and water, wherein the pH is from 4.8 to 5.9 and wherein the concentration of dalbavancin is in the range of 15 mg/ml to 22 mg/ml.

In an aspect, the aqueous composition of dalbavancin consists essentially of dalbavancin or a pharmaceutically acceptable salt of dalbavancin, an osmolality adjusting agent and water, wherein the pH is from 4.8 to 5.9 and wherein the concentration of dalbavancin is in the range of 1 mg/ml to 25 mg/ml.

In an aspect, the aqueous composition of dalbavancin consists essentially of dalbavancin or a pharmaceutically acceptable salt of dalbavancin, an osmolality adjusting agent and water, wherein the pH is from 4.8 to 5.9 and wherein the concentration of dalbavancin is in the range of 1 mg/ml to 7 mg/ml.

In an aspect, the aqueous composition of dalbavancin consists essentially of dalbavancin or a pharmaceutically acceptable salt of dalbavancin, an osmolality adjusting agent and water, wherein the pH is from 4.8 to 5.9 and wherein the concentration of dalbavancin is in the range of 15 mg/ml to 22 mg/ml.

In an aspect, the aqueous composition of dalbavancin consists essentially of dalbavancin or a pharmaceutically acceptable salt of dalbavancin, an osmolality adjusting agent, one or more amino acid and water, wherein the pH is from 4.8 to 5.9 and wherein the concentration of dalbavancin is in the range of 1 mg/ml to 25 mg/ml.

In an aspect, the aqueous composition of dalbavancin consists essentially of dalbavancin or a pharmaceutically acceptable salt of dalbavancin, an osmolality adjusting agent, one or more amino acid and water, wherein the pH is from 4.8 to 5.9 and wherein the concentration of dalbavancin is in the range of 1 mg/ml to 7 mg/ml.

In an aspect, the aqueous composition of dalbavancin consists essentially of dalbavancin or a pharmaceutically acceptable salt of dalbavancin, an osmolality adjusting agent, a stabilizing agent and water, wherein the pH is from 4.8 to 5.9 and wherein the concentration of dalbavancin is in the range of 15 mg/ml to 22 mg/ml.

In an aspect, the aqueous composition of dalbavancin consists of dalbavancin or a pharmaceutically acceptable salt of dalbavancin, an osmolality adjusting agent and water, wherein the pH is from 4.8 to 5.9 and wherein the concentration of dalbavancin is in the range of 1 mg/ml to 25 mg/ml.

In an aspect, the aqueous composition of dalbavancin consists of dalbavancin or a pharmaceutically acceptable salt of dalbavancin, an osmolality adjusting agent and water, wherein the pH is from 4.8 to 5.9 and wherein the concentration of dalbavancin is in the range of 1 mg/ml to 7 mg/ml.

In an aspect, the aqueous composition of dalbavancin consists of dalbavancin or a pharmaceutically acceptable salt of dalbavancin, an osmolality adjusting agent and water, wherein the pH is from 4.8 to 5.9 and wherein the concentration of dalbavancin is in the range of 15 mg/ml to 22 mg/ml.

In an aspect, the aqueous composition of dalbavancin consists of dalbavancin or a pharmaceutically acceptable salt of dalbavancin, an osmolality adjusting agent, one or more amino acid and water, wherein the pH is from 4.8 to 5.9 and wherein the concentration of dalbavancin is in the range of 1 mg/ml to 25 mg/ml.

In an aspect, the aqueous composition of dalbavancin consists of dalbavancin or a pharmaceutically acceptable salt of dalbavancin, an osmolality adjusting agent, a stabilizing agent and water, wherein the pH is from 4.8 to 5.9 and wherein the concentration of dalbavancin is in the range of 1 mg/ml to 7 mg/ml.

In an aspect, the aqueous composition of dalbavancin consists of dalbavancin or a pharmaceutically acceptable salt of dalbavancin, an osmolality adjusting agent, one or more amino acid and water, wherein the pH is from 4.8 to 5.9 and wherein the concentration of dalbavancin is in the range of 15 mg/ml to 22 mg/ml.

In an aspect, the aqueous dalbavancin formulation has less than an 8% increase of mannosyl aglycon impurity as measured by HPLC.

In an aspect, the aqueous dalbavancin formulation has less than a 4% increase of mannosyl aglycon impurity as measured by HPLC after storage at room temperature for 6 months.

In an aspect, the aqueous dalbavancin formulation has less than a 2% increase of mannosyl aglycon impurity as measured by HPLC after storage at room temperature for 3 months.

In an aspect, the formulations mentioned above can be used for treatment of adult and pediatric patients with acute bacterial skin and skin structure infections (ABSSSI) caused by designated susceptible strains of Gram-positive microorganisms.

### EXAMPLES (METHODS, RESULTS, DISCUSSION)

After preparation, the initial time point level of active pharmaceutical ingredient and impurities were determined by high performance liquid chromatography (HPLC) and afterwards containers were loaded into stability chambers at different storage conditions, 40° C. and 25° C.

In order to determine the stability of active pharmaceutical ingredient in formulations according to the present disclosure, containers were taken from stability chambers at various time points, such as 28 days, 1 month, 2 months, 3 months, 6 months etc. and analyzed by HPLC.

The assay for active pharmaceutical ingredient was determined by a gradient HPLC method using internal standards and a DAD detector. Impurities were determined by using the same HPLC method with amount determined by area percentage. Parallel to assay and impurities, component distribution is determined for the five main homologues (A0, A1, B0, B1 and B2) also as an area percentage method. In all cases, a reverse-phase C18 column was used.

### HPLC Assay and Impurities Method:

Mobile phase A: mixture phosphate buffer and acetonitrile (85/15)
Mobile phase B: mixture phosphate buffer and acetonitrile (35/65)
Mode: LC
Detector: UV 230 nm.
Column: 2.1×5 mm; 3.5 μm packing
Column temperature: 45° C.

Samples are prepared by dilution to 0.3 mg/ml. Every sample sequence is run with injections of internal impurities ID standards as with repeated injections of blank diluent solution. The system is regularly washed because of possible back-pressure increases.

Labeled amount of dalbavancin in percentage is calculated:

$$Assay \% = ( \frac{{A}_{s}}{{A}_{std}} ) \times ( \frac{{c}_{std}}{{c}_{s}} ) \times 100$$

Aₛ—peak response of dalbavancin (as a sum of five homologues) from the sample solution
A_{std}—peak response of dalbavancin (as a sum of five homologues) from the standard solution
c_{std}—concentration of dalbavancin in the standard solution (mg/mL)
cₛ—nominal concentration of dalbavancin in the sample solution (mg/mL)

The content of impurities is given as area % of the total area, calculated using the following equation:
Aᵢ—peak response of an impurity from the sample solution.
Aₜₒₜ—sum of all peak responses from the sample solution.

$$Hom \% = ( \frac{{A}_{h}}{{A}_{d}} ) \times 100$$

Aₕ—peak response of a homologue from the sample solution.
A_{d}—sum of all homologue peak responses (A0, A1, B0, B1 and B2) from the sample solution.

### Preparation of Exemplary Formulation:

Water for Injection (90% of final volume) was added into a vessel and stirring was started.

The excipients required for the different formulations were added. Dalbavancin hydrochloride was added in an amount to get a final concentration of 5 mg/ml. The solution was stirred until all dry components were dissolved.

pH was adjusted to target pH value by adding in the needed amount of HCl or NaOH (1M solution). Additional Water for Injection was added into the solution to reach final volume. The solution was filtrated through 0.22 μm filter and filled in containers such as glass vials or plastic bags.

### Example 1

This example shows the change in mannosyl aglycon impurity in a formulation of 5 mg/ml of dalbavancin hydrochloride in Water for Injection at different pHs at two different storage conditions.

TABLE 2
Change in mannosyl aglycon impurity if formulation
at different pH and different storage conditions.

| | Time | | Δ mannosyl aglycon | |
|---|---|---|---|---|
| pH | point | ° C. | impurity/% | Apperance |
| 3 | 3 months | 25 | 4.3 | clear solution, no |
| | 6 months | 25 | 7.8 | particles |
| 4 | 3 months | 25 | 2.3 | clear solution, no |
| | 6 months | 25 | 4.3 | particles |
| 5 | 3 months | 25 | 1.9 | clear solution, no |
| | 6 months | 25 | 3.3 | particles |
| 5.5 | 3 months | 25 | 0.2 | clear solution, no |
| | 6 months | 25 | 0.5 | particles |
| 3 | 4 weeks | 40 | 10.2 | clear solution, no |
| | | | | particles |
| 4 | 4 weeks | 40 | 5.7 | clear solution, no |
| | | | | particles |
| 5 | 4 weeks | 40 | 3.9 | clear solution, no |
| | | | | particles |
| 5.5 | 4 weeks | 40 | 0.8 | clear solution, no |
| | | | | particles |

### Example 2

This example shows the change in total impurities in formulations of 5 mg/ml of dalbavancin hydrochloride in 5% dextrose after 3 months and 6 months storage at 25° C. The two formulations have a shift in pH of 0.3 pH units and 0.5 pH units respectively.

TABLE 3
Change in total impurities in two formulations
with different shift in pH

| | | | |
|---|---|---|---|
| Time | | Δ total | |
| point | pH | impurities | Apperance |

Formulation with dextrose with 0.3 units pH shift

| | | | |
|---|---|---|---|
| Start | 5.9 | 0.00 | clear solution, no visible particles |
| 3 months | 5.6 | 0.99 | clear solution, no visible particles |
| 6 months | 5.6 | 1.64 | clear solution, no visible particles |

Formulation with dextrose with 0.5 units pH shift

| | | | |
|---|---|---|---|
| Start | 5.7 | 0.00 | clear solution, no visible particles |
| 2 months | 5.2 | 0.83 | clear solution, no visible particles |
| 6 months | 5.2 | 2.30 | clear solution, no visible particles |

### Example 3

This example shows the change in total impurities in a formulation of 5 mg/ml of dalbavancin hydrochloride in 5% dextrose after 3 months and 6 months storage at 25° C. The example includes formulations with only dextrose, with 10 mM histidine, with 10 mM aspartic, 10 mM of L-glutamic acid, 10 mM of L-isoleucine acid and 10 mM of succinic acid.

TABLE 4
Change in total impurities in formulations
with different L-amino acids.

| | | | |
|---|---|---|---|
| Time | | Δ total | |
| point | pH | impurities | Apperance |

Formulation with only dextrose (exp no 073)

| | | | |
|---|---|---|---|
| Start | 5.7 | 0.00 | clear solution, no visible particles |
| 3 months | 5.2 | 0.95 | clear solution, no visible particles |
| 6 months | 5.2 | 2.01 | clear solution, no visible particles |

Formulation with dextrose and 10 mM of L-histidine (exp no 111)

| | | | |
|---|---|---|---|
| Start | 5.7 | 0.00 | clear solution, no visible particles |
| 3 months | 5.6 | 0.74 | clear solution, no visible particles |
| 6 months | 5.5 | 1.24 | clear solution, no visible particles |

Formulation with dextrose and 10 mM of L-aspartic acid (exp no 068)

| | | | |
|---|---|---|---|
| Start | 5.8 | 0.00 | clear solution, no visible particles |
| 3 months | 5.5 | 0.68 | clear solution, no visible particles |
| 6 months | 5.5 | 1.81 | clear solution, no visible particles |

Formulation with dextrose and 10 mM of L-glutamic acid (exp no 084)

| | | | |
|---|---|---|---|
| Start | 5.7 | 0.00 | clear solution, no visible particles |
| 3 months | 5.5 | 1.18 | clear solution, no visible particles |

Formulation with dextrose and 10 mM of L-isoleucine (exp no 089)

| | | | |
|---|---|---|---|
| Start | 5.7 | 0.00 | clear solution, no visible particles |
| 3 months | 5.3 | 1.20 | clear solution, no visible particles |

Formulation with dextrose and 10 mM of succinic acid

| | | | |
|---|---|---|---|
| Start | 5.4 | N/A | white suspension immediately, |
| | | | white precipitate after 24 h |

### Example 4

This example shows the change in mannosyl aglycon impurity in a formulation of 20 mg/ml of dalbavancin hydrochloride in 5% dextrose solution at different pHs at two different storage conditions.

TABLE 5
Change in mannosyl aglycon impurity in formulation
with 20 mg/ml of dalbavancin.

| pH | Time point | ° C. | Δ mannosyl aglycon impurity/% |
|---|---|---|---|
| 4 | 4 weeks | 40 | 9.55 |
| 4 | 4 weeks | 25 | 1.24 |
| 5 | 4 weeks | 40 | 5.94 |
| 5 | 4 weeks | 25 | 0.7 |

### Example 5

The following numbered items represent embodiments of liquid pharmaceutical formulations comprising active component.

Item 1. An aqueous dalbavancin formulation, comprising dalbavancin or a pharmaceutically acceptable salt of dalbavancin, and optionally an osmolality adjusting agent; wherein the formulation has a pH in the range of 4.8 and 5.9.

Item 2. An aqueous dalbavancin formulation, consisting of dalbavancin or a pharmaceutically acceptable salt of dalbavancin and an osmolality adjusting agent; wherein the formulation has a pH in the range of 4.8 and 5.9.

Item 3. An aqueous dalbavancin formulation, comprising dalbavancin or a pharmaceutically acceptable salt of dalbavancin, an osmolality adjusting agent and optionally a pH stabilizing agent; wherein the formulation has a pH in the range of 4.8 and 5.9.

Item 4. An aqueous dalbavancin formulation, comprising dalbavancin or a pharmaceutically acceptable salt of dalbavancin, an osmolality adjusting agent and at least one amino acid; wherein the formulation has a pH in the range of 4.8 and 5.9.

Item 5. An aqueous dalbavancin formulation, consisting of dalbavancin or a pharmaceutically acceptable salt of dalbavancin, an osmolality adjusting agent and at least one amino acid; wherein the formulation has a pH in the range of 4.8 and 5.9.

Item 6. The aqueous dalbavancin formulation according to items 1 to 5, wherein the osmolality adjusting agent is dextrose.

Item 7. The aqueous dalbavancin formulation according to items 4 and 5, wherein the amino acid is an L-amino acid.

Item 8. The aqueous dalbavancin formulation according to item 7, wherein the amino acid is L-histidine.

Item 9. The aqueous dalbavancin formulation according to item 7, wherein the amino acid is L-glutamic acid.

Item 10. The aqueous dalbavancin formulation according to item 7, wherein the amino acid is L-isoleucine.

Item 11. The aqueous dalbavancin formulation according to items 1 to 10, wherein the concentration of dalbavancin is in the range of 1 mg/ml to 25 mg/ml.

Item 12. The aqueous dalbavancin formulation according to item 11, wherein the concentration of dalbavancin is in the range of 1 mg/ml to 7 mg/ml.

Item 13. The aqueous dalbavancin formulation according to item 11, wherein the concentration of dalbavancin is in the range of 15 mg/ml to 22 mg/ml.

Item 14. The aqueous dalbavancin formulation according to item 11, wherein the concentration of dalbavancin is in the range of 3 mg/ml to 6 mg/ml.

Item 15. The aqueous dalbavancin formulation according to item 11, wherein the concentration of dalbavancin is in the range of 18 mg/ml to 22 mg/ml.

Item 16. The aqueous dalbavancin formulation according to items 1 to 15, wherein the pH is in the range of 5.0 to 5.9.

Item 17. The aqueous dalbavancin formulation according to items 1 to 15, wherein the pH is in the range of 5.2 to 5.9.

Item 18. The aqueous dalbavancin formulation according to items 1 to 15, wherein the pH is in the range of 5.3 to 5.9.

Item 19. The aqueous dalbavancin formulation according to items 1 to 18, wherein the formulation has less than an 8% increase of mannosyl aglycon impurity as measured by HPLC.

Item 20. The aqueous dalbavancin formulation according to items 1 to 18, wherein the solution has less than a 4% increase of mannosyl aglycon impurity as measured by HPLC after storage at room temperature for 3 months.

Item 21. The aqueous dalbavancin formulation according to items 1 to 20, wherein the solution has less than a 2% increase of mannosyl aglycon impurity as measured by HPLC after storage at room temperature for 3 months.

Item 22. The aqueous dalbavancin formulation according to items 1 to 20, wherein the pH shift is not more than 1.0 pH unit after storage at room temperature for at least 6 months.

Item 23. The aqueous dalbavancin formulation according to item 22, wherein the pH shift is not more than 0.8 pH units after storage at room temperature for at least 6 months.

Item 24. The aqueous dalbavancin formulation according to item 22, wherein the pH shift is not more than 0.6 pH units after storage at room temperature for at least 6 months.

Item 25. The aqueous dalbavancin formulation according to items 1 to 24 is for parenteral use.

Item 26. A method of treatment of an acute bacterial infection caused by Staphylococcus aureus, Streptococcus pyogenes, Streptococcus agalactiae, Streptococcus dysgalactiae, Streptococcus anginosus group or vancomycin susceptible isolates of Enterococcus faecalis comprising the step of administering the aqueous solution as defined in items 1 to 25 to a patient in need thereof.

Item 27. The aqueous dalbavancin formulation as defined in items 1 to 25 for use in treatment of acute bacterial infections caused by Staphylococcus aureus, Streptococcus pyogenes, Streptococcus agalactiae, Streptococcus dysgalactiae, Streptococcus anginosus group or vancomycin susceptible isolates of Enterococcus faecalis.

1-12. (canceled)

13. A ready to administer aqueous dalbavancin formulation, comprising
1 mg/ml to 7 mg/ml of dalbavancin or a pharmaceutically acceptable salt thereof,
one or more amino acids,
a pH adjusting agent to adjust the pH to the range of 4.8 to 5.9, and
optionally an osmolality adjusting agent,
wherein the formulation is a clear solution with no visible particles after storage at room temperature for at least 6 months.

14. The ready to administer aqueous dalbavancin formulation of claim 13, wherein the one or more amino acids is selected from L-histidine, L-aspartic acid, L-glutamic acid, and L-isoleucine.

15. The ready to administer aqueous dalbavancin formulation of claim 13, wherein the osmolality adjusting agent is dextrose.

16. The ready to administer aqueous dalbavancin formulation of claim 13, wherein the osmolality of the formulation is 270 to 340 mOsmol/kg.

17. The ready to administer aqueous dalbavancin formulation of claim 13, wherein a pH shift of the formulation is not more than 1.0 pH units after storage at room temperature for at least 6 months

18. The ready to administer aqueous dalbavancin formulation of claim 13, wherein a pH shift of the formulation is not more than 0.8 pH units after storage at room temperature for at least 6 months.

19. The ready to administer aqueous dalbavancin formulation of claim 13, wherein the solution has less than a 4% increase of mannosyl aglycon impurity as measured by HPLC after storage at room temperature for 3 months.

20. The ready to administer aqueous dalbavancin formulation of claim 13, wherein the solution has less than a 2% increase of mannosyl aglycon impurity as measured by HPLC after storage at room temperature for 3 months
